(19) United States
(12) Patent Application Publication
(10) Pub. No.: US 2024/0165074 A1
(43) Pub. Date: May 23, 2024

(54) COMPOSITION OF THE PHYTOCOMPOUNDS INTENDED TO ENHANCE CYTOTOXIC ACTIVITY IN THE TREATMENT OF ACUTE LYMPHOBLASTIC LEUKEMIA

(71) Applicants: GDANSKI UNIWERSYTET MEDYCZNY, Gdansk (PL); FUNDACJA GETRESPONSE CARES, Gdansk (PL)

(72) Inventors: Natalia MIEKUS-PURWIN, Gdynia (PL); Patrycja KOSZALKA, Gdansk (PL); Grzegorz STASILOJC, Gdansk (PL); Maciej NIEDZWIECKI, Gdansk (PL); Tomasz BACZEK, Gdansk (PL); Szymon GRABOWSKI, Gdynia (PL)

(21) Appl. No.: 18/549,124

(22) PCT Filed: Feb. 10, 2022
(86) PCT No.: PCT/PL2022/050006

§ 371 Date: Sep. 5, 2023

(30) Foreign Application Priority Data
Mar. 11, 2021 (PL) P.437273

(51) Int. Cl. A61K 31/353; A61K 31/05; A61K 31/12; A61P 35/00

(52) U.S. Cl. CPC A61K 31/353; A61K 31/05; A61K 31/12; A61P 35/00

(57) ABSTRACT

The invention concerns a mix of the following phytocompounds: curcumin, genistein, resveratrol, quercetin applied to enhance cytotoxic activity in the treatment of acute lymphoblastic leukemias.

### FIELD OF THE INVENTION

The invention concerns a new mix of phytocompounds, i.e. resveratrol, quercetin, genistein, and curcumin, applied to supplement chemotherapy in acute lymphoblastic leukemia.

### BACKGROUND OF THE INVENTION

According to the current World Health Organisation classification, acute lymphoblastic leukemia (ALL) is defined as precursor cell lymphoid neoplasm. Because of the heterogenous nature of the disease, oncologists need to run many additional tests such as, e.g., the test for the presence of the BCR-ABL transcript which is an adverse prognostic factor, or identification of the immunological subtype. ALL is the most frequent malignant neoplasm in children, where therapeutic failures and recurrences affect nearly 20% of all patients. In view of the recurrence of the disease in some children and their resistance to the administered steroid therapy and chemotherapy it is important to develop new, more effective and less toxic therapeutic methods.

The desire to propose improved schemes for the treatment of children suffering from high-risk ALL was the starting point of the research taken up in the project. The research focused on the science of natural substances, which is an area not fully explored in oncology, and the anti-cancer potential of those substances in children with ALL. Scientific research rarely focuses on subjects of the paediatric group. Therefore, based on the insights gained in the research oriented on adult patients suffering from neoplasms of other types, the presented research verified anti-cancer effects of plant-origin compounds such as: curcumin, resveratrol, quercetin, and genistein on the ALL cell lines. The compounds, either taken as individual substances, or in combinations of two compounds, have already been verified for their anti-cancer effects, but the combination of all four compounds is a single mix has never been subject to research in ALL leukemias.

The selected compounds have not only been confirmed to demonstrate anti-cancer properties, but administration of a mix of the phytocompounds has enabled avoiding the problem of bioavailability (i.e. the attainment of therapeutic concentrations in the organism) linked to clinical application of natural compounds.

The combination according to the invention demonstrates a strong synergistic effect and carries a lesser, prevailingly temporary effect on the healthy cells. The research has revealed that even at low doses genistein and curcumin act synergistically against the neoplastic cells of the ALL. Moreover, using the mix made up of resveratrol and quercetin only did not demonstrate any significant action on the MOLT-4 cells, whereas the combination of the two phytochemicals with genistein and curcumin enhanced their anti-cancer effects. It has been confirmed that only administration of the phytocompound mix composed of the four analysed substances did, within the tested concentration range, result in anti-cancer effects without permanent negative impact on the control cells (fibroblasts).

The discovery of the synergistic anti-cancer effect on the ALL cell lines paved the way for further research on the application of the phytochemical mix in paediatric patients suffering from ALL. The research initiated by the authors of this invention can, in the future, contribute to changing the therapeutic schemes in patients of the high-risk group to schemes that will be more effective, less toxic, and acceptable to the infirm.

Comparative research of the anti-cancer effects of individual phytochemicals and their mixes has not been undertaken on the ALL cell lines to date, and therefore in vitro tests were performed for the individual compounds and their combinations in terms of their action on the researched leukemia cells (the MOLT-4 cell line). With the BJ cell line applied alongside (the control line of normal cells of limited number of divisions) the toxicity of the selected compounds on cancer-non-affected cells (fibroblasts) has also been verified. Today, scientific reports focus almost exclusively on testing phytochemicals on neoplastic cell lines without conducting parallel experiments on normal cells. Thanks to the research conducted both on neoplastic and normal cells, the authors have assessed the anti-cancer potential of the tested mix whilst simultaneously assessing its potential negative effects on normal cells. By applying various concentrations of the selected compounds within their bioassimilability ranges, the dose of the tested phytochemicals which needs to be administered to attain the chemotherapeutic effect without permanently damaging the healthy cells has been verified.

It is a known fact that radiotherapy or chemotherapy damage or weaken the patient's immunological resistance, a paediatric patient in particular. That is why a search is continuing for an appropriate composition that will strengthen the immune system in cancer treatment.

### SUMMARY OF THE INVENTION

The invention concerns a composition of the mix of the following phytocompounds: curcumin, genistein, resveratrol, quercetin applied to enhance cytotoxic activity in the treatment of acute lymphoblastic leukemia.

The composition where the cytotoxic activity of individual compounds in the blood is: 2.25 μg/ml for curcumin, 3 μg/ml for genistein, 1.5 μg/ml for quercetin, and 0.5 μg/ml for resveratrol.

The composition is intended for the paediatric patient, administered orally.

The terms used above and in the patent description and claims means:
Paediatric patient—a patient up to 18 years of age.
Phytocompounds—substances/compounds of plant origin, contained in colour fruit and vegetables, though not of the vitamin or mineral groups.
Phytochemicals—synonym for phytocompounds.
GCQR—the mix of genistein, curcumin, quercetin, and resveratrol.

### BRIEF DESCRIPTION OF THE DRAWINGS

The object of the invention is illustrated in the drawings, in which:

FIG. 1 shows a changes in the percentage of viable neoplastic cells of the MOLT-4 line treated with individual phytocompounds. The decrease in their viability is significant for curcumin and even more prominent for genistein. The concentrations specified as 0.5×, 1×, and 2× define the concentration of the specific phytocompound in correlation with its bioavailability in vivo, where 1× represents the maximum bioavailability level of the specific compound in the blood (2.25 μg/ml for curcumin, 3 μg/ml for genistein, 1.5 μg/ml for quercetin, and 0.5 μg/ml for resveratrol). Viability was measured under the MTT assay for the times of 24-, 48-, and 72-hours following administration of the analysed compounds. Control (C) is defined as 100% viability which reflects the viability of the population not treated with phytocompounds at each time point. All points present the average results of 3 independent tests plus standard deviations *P<0.05, **P<0.01, ***P<0.001 in relation to the control. The lines depict the differences between the two groups, ##P<0.01, ###P<0.001.

FIG. 2 shows a changes in the percent share of viable neoplastic cells of the MOLT-4 line and normal BJ fibroblasts treated with selected combinations of the phytocompounds. The combination of curcumin and genistein substantially enhances the effect of decreased viability of the MOLT4 cells, and with resveratrol and quercetin added to the combination, the effect is substantially enhanced and the dose needed to attain the effect is reduced. Genistein alone and any analysed combination, do not decrease the viability of the BJ cells. The concentrations specified as 0.5×, 1×, and 2× define the concentration of the specific phytocompound in correlation with its bioavailability in vivo, where 1× represents the maximum bioavailability level of the specific compound in the blood (2.25 μg/ml for curcumin, 3 μg/ml for genistein, 1.5 μg/ml for quercetin, and 0.5 μg/ml for resveratrol). Viability was measured under the MTT assay for the times of 24-, 48-, and 72-hours (MOLT-4), or 24- and 72-hours (BJ) following administration of the analysed compounds. Control (C) is defined as 100% viability which reflects the viability of the population not treated with phytocompounds at each time point. All points present the average results of 3 independent tests plus standard deviations *P<0.05, **P<0.01, ***P<0.001 in relation to the control. The lines depict the differences between the two groups, #P<0.05, ##P<0.01, ###P<0.001.

FIG. 3 shows a changes in absolute viability of the neoplastic cells of the MOLT-4 line treated with selected combinations of the phytocompounds. In the case of the in vivo bioavailable dose, 1×, only the combination of all 4 phytocompounds was able to effectively inhibit the growth of the neoplastic cell population within 48 hours following administration of the phytocompounds. The concentrations specified as 0.5×, 1×, and 2× define the concentration of the specific phytocompound in correlation with its bioavailability in vivo, where 1× represents the maximum bioavailability level of the specific compound in the blood (2.25 μg/ml for curcumin, 3 μg/ml for genistein, 15 μg/ml for quercetin, and 0.5 μg/ml for resveratrol). Viability was measured under the MTT assay for the times of 24-, 48-, and 72-hours (MOLT-4), or 24- and 72-hours (BJ) following administration of the analysed compounds. Control (C) is defined as 100% viability which reflects the viability of the population not treated with phytocompounds at the 24-hours' time point. All points present the average results of 3 independent tests plus standard deviations *P<0.05, **P<0.01, ***P<0.001 in relation to the control.

FIG. 4 shows a changes in the mitochondrial membrane potential (MMP) of the neoplastic cells of the MOLT-4 line and normal BJ cells treated with selected combinations of the phytocompounds. For MOLT-4 cells, the phytocompound mixture reduced the MMP to a statistically significant extent, regardless of the dose used, and the maximum decrease was reached at the dose of 2× bioassimilability. For BJ cells, significant decreases were observed for 1× and 2× doses, although the maximum decrease in MMP levels was not observed until 24 hours after induction with the 1× dose, and 24 and 48 hours after induction with the 2× dose. The concentrations specified as 0.5×, 1×, and 2× define the concentration of the specific phytocompound in correlation with its bioavailability in vivo, where 1× represents the maximum bioavailability level of the specific compound in the blood (2.25 μg/ml for curcumin, 3 μg/ml for genistein, 1.5 μg/ml for quercetin, and 0.5 μg/ml for resveratrol). The MMP measurements were taken with a flow cytometer with the applied fluorescent dye, JC-1 (1,1′,3,3′-tetraethyl-5,5′,6,6′-tetrachloroimidacarbocyanine iodide) for the times of 24-, 48-, and 72-hours. Control (C) reflects the initial MMP level for the cell population not treated with the phytocompounds at each time point. All points present the average results of 3 independent tests performed in 3 technical repetitions for 10,000 cells per repetition, plus standard deviations *P<0.05, ***P<0.001 in relation to the control.

FIG. 5 shows a changes in cell membrane permeability of the neoplastic cells of the MOLT-4 cell line and the normal BJ cells treated with selected combinations of the phytocompounds. For both tested cell lines, the mix of the phytocompounds at the 2× dose increased the number of cells demonstrating higher permeability of the cell membrane. For the neoplastic cell line, also doses 0.5× and 1× caused a statistically significant increase in the number of cells demonstrating higher permeability of the cell membrane compared to the control cells. As for the population of BJ cells, the number of cells with higher membrane permeability increased to insignificant levels for both the 0.5× and 1× doses.

The concentrations specified as 0.5×, 1×, and 2× define the concentration of the specific phytocompound in correlation with its bioavailability in vivo, where 1× represents the maximum bioavailability level of the specific compound in the blood (2.25 μg/ml for curcumin, 3 μg/ml for genistein, 1.5 μg/ml for quercetin, and 0.5 μg/ml for resveratrol).

### DETAILED DESCRIPTION OF THE INVENTION

### Description of Embodiments

The present invention is illustrated in the following embodiments which do not limit the invention.

### Examples

Assays were performed so as to assess the anti-cancer effects of the selected compounds of natural origin on ALL cells:

1. Viability Test Comprising the Use of a Test Cell Line (Cultures in the Presence of Individual Phytochemicals or their Mix) and the Control Line

The test is based on an assessment of the enzymatic activity of mitochondria using MTT, 3-(4,5-Dimethylthiazol-2-yl)-2,5-diphenyltetrazolium) bromide. Live and metabolically active cells reduce MTT to formazan crystals. In the test, a 96-well plate was seeded with 3×10+ cells per well in the volume of 50 μl of standard culture medium, followed by the addition of serial dilutions of the analysed compounds in the volume of 50 μl of the culture medium to obtain the final volume of 100 μl. An identical quantity of the solvent without the analysed phytocompounds was added to the control wells. The phytocompounds were stored dissolved in 100% DMSO, and therefore each dilution and the culture media added for control were supplemented with DMSO up to the non-toxic final concentration of 0.05%. The viability of the cells was assessed after a specific incubation time (24-, 48-, or 72-hours) by adding 20 μl of the yellow MTT solution to each well, and the colorimetric conversion of MTT into purple formazan crystals was assessed after 2-3 hours of incubation by dissolving the crystals with acidic isopropanol and taking a spectrophotometric measurement at the wavelength of 570 nm.

The measurements of cell viability with the application of this assay were performed on the following lines: MOLT-4 (the line of human ALL leukemia, a standard cell line used to assess the effects of pharmaceutical compounds on this type of leukemias) and BJ (human foreskin fibroblasts, primary cells grown in a short-term culture).

The effects of the analysed phytocompounds were assessed in correlation with their bioavailability in vivo, using doses 0.5×, 1×, and 2×, where 1× was the maximum level of bioavailability of a specific compound in the blood (2.25 μg/ml for curcumin, 3 μg/ml for genistein, 1.5 μg/ml for quercetin and 0.5 μg/ml for resveratrol).

### 2. Analysis of the Mitochondrial Membrane Potential by Flow Cytometry

The analysis was based on assaying the fluorescence of the JC-1 dye (1,1′,3,3′-tetraethyl-5,5′,6,6′-tetrachloroimidacarbocyanine iodide) added to the cell suspension. CCCP (carbonyl cyanide m-chlorophenyl hydrazone), which uncoupled the mitochondria and prevented JC-1 aggregation was used as a low-potential control. The cells not treated with phytochemicals or their mixes served as a control of high mitochondrial membrane potential.

### 3. Analysis of Cell Membrane Permeability by Flow Cytometry

Assessment of cell membrane permeability was based on the phenomenon of the Trypan blue dye (3Z, 3′Z)-3,3′-[(3,3′-dimethylbiphenyl-4,4′-diyl)didiazene-2,1-diyl]bis(5-amino-4-hydroxynaphthalene-2,7-disulfonate) penetration into cells with the compromised cell membrane. Cells not treated with the phytochemicals or mixtures of the phytochemicals were used as control (cells with continuous membranes). The result was expressed as a percent of the population demonstrating higher permeability of the cell membrane.

The measurement of membrane continuity was taken using a flow cytometer and a dye of fluorescent properties, namely Trypan blue after 24, 48, and 72 hours. The control (C) reflects the initial fluorescence level corresponding to the population of cells not treated with phytocompounds at each time point. All points represent the mean of the results for 3 independent assays performed in 3 technical replicates for 10,000 cells in each replicate, and standard the deviations ***P<0.001 in relation to the control.

### The Findings:

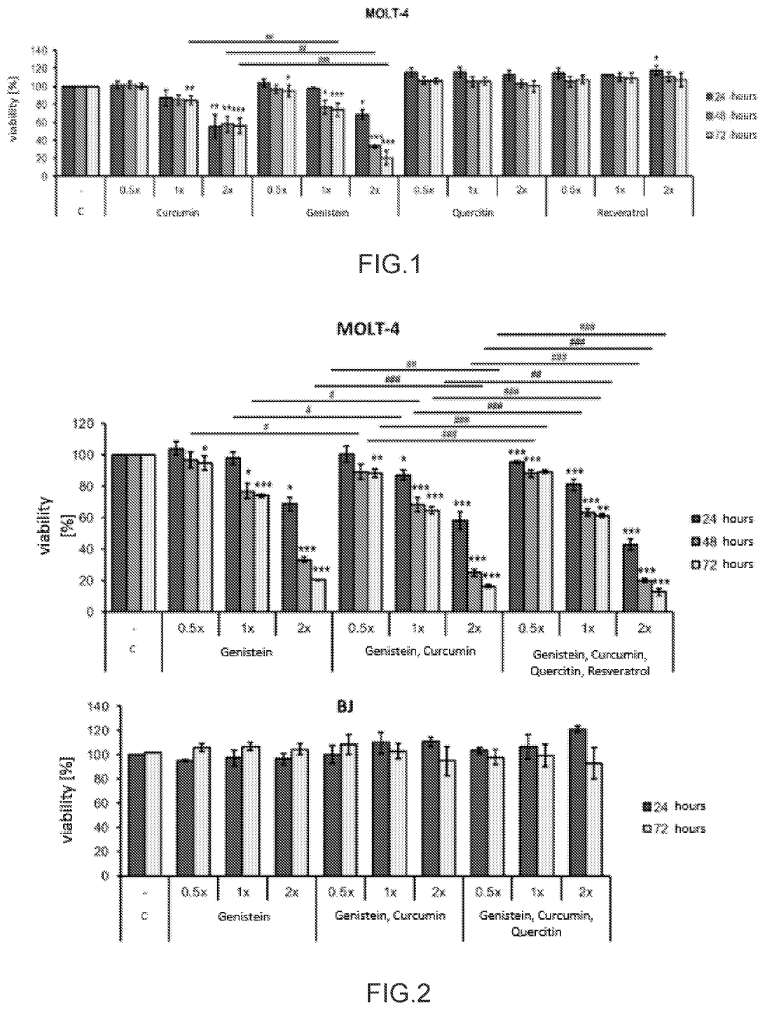

The initial analyses of the phytocompounds (FIG. 1) showed that when administered at the bioavailable dose, only genistein reduced the viability of the MOLT-4 neoplastic cells after 48 hours, while curcumin administered at the identical dose needed 72 hours to carry the same effect. Quercetin and resveratrol, on the other hand, did not demonstrate any impact on the viability of those cells, even at doses that were attainable exclusively in vitro (2×), where in the case of genistein and curcumin the doses proved to be relatively highly cytotoxic.

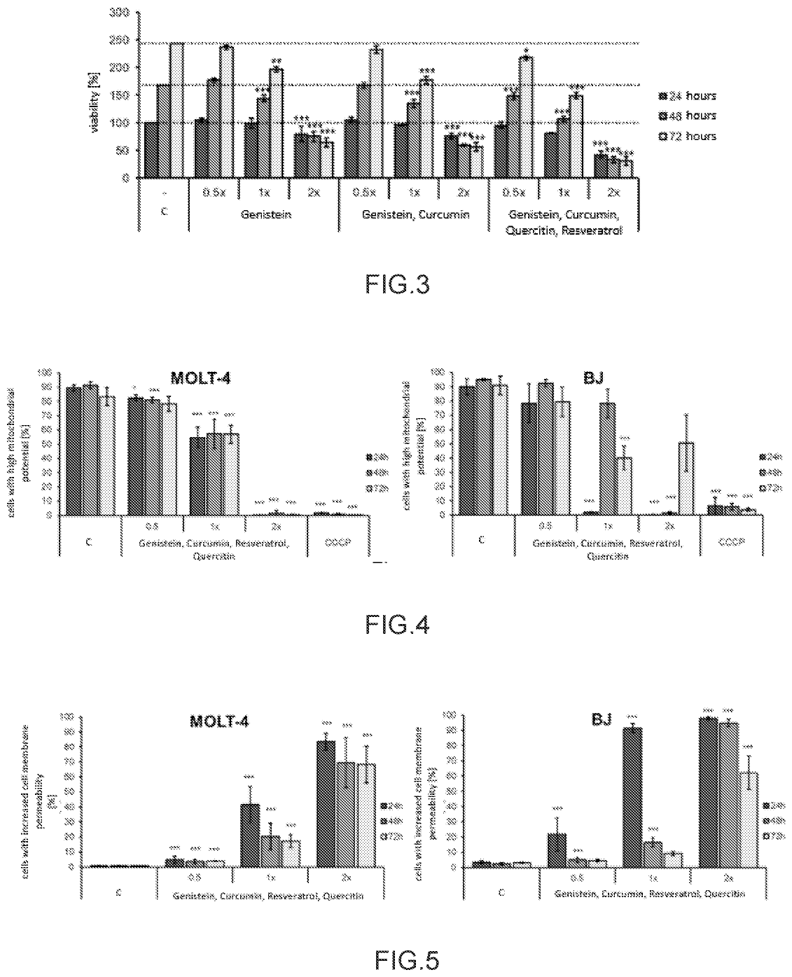

Subsequently, various combinations of the phytocompounds were analysed, with two to three phytocompounds per combination, as well as the combination of all four analysed compounds. In the case of a two-compound combination, curcumin mixed with genistein in the same doses gave the strongest effect (FIG. 2). In the case of the bioavailable dose, enhancement of the effect was already visible after 24 hours and continued for up to 48 hours; the biological effect was also successfully attained after 48 hours at the dose of 0.5× which, for a single compound, was only effective in the case of genistein after 72 hours. In the case of doses bioavailable in vivo, the enhancement lasted for up to 48 hours following administration of the compounds, while for higher doses attainable in vitro the effect continued for up to 72 hours. With resveratrol and quercetin added, the mix (GCQR) demonstrated substantially higher cytotoxic effects as early as after 24 hours for all doses (FIG. 2). For all doses, the effect was sustained after 48 hours, while for doses 1× and 2× the effect was also observed after 72 hours. This demonstrates a significant enhancement of the effects of the phytocompounds even if administered in doses at the range bioavailable in vivo. In addition, an analysis of the absolute growth of the culture (FIG. 3) showed that the GCQR mix at the dose bioavailable in vivo (1×) is able to effectively block the growth of the MOLT-4 cell population in 48 hours following its administration.

An assessment of the potential cytotoxic effect in the case of healthy cells of the organism carried out with an application of the normal foetal BJ fibroblasts typically used in analyses of such type demonstrated that the GCQR mix does not affect the viability of the BJ cells, even at a dose twice higher than the dose bioavailable in vivo (2×). Hence, it potentially has no toxic effect on non-neoplastic cells of the organism (FIG. 2).

A decrease of the mitochondrial membrane potential (MMP) was observed for both MOLT-4 neoplastic cells and normal BJ cells (FIG. 4). Both cell lines were treated with a combination of the phytocompounds (curcumin, genistein, quercetin and resveratrol). Compared to the control, i.e. non-treated cells of each cell line, differences in the decrease of MMP were observed between the MOLT-4 neoplastic cells and normal BJ cells. For the MOLT-4 cells, a statistically significant decrease of the mitochondrial membrane potential was observed after 24 h for all used concentrations of the phytocompound combination, and the decrease depended on the administered dose. The MMP decrease reached the maximum level for the 2× bioavailable dose at all observed time points. These results demonstrate a substantial impairment of the functionality of these cells, particularly of the cells treated with the 1× and 2× bioavailable doses as the result of the maximally impaired capability to produce×ATP by the cell's mitochondria. The phenomenon is also reflected in the results which demonstrate an increased permeability of the cell membrane in this cell line at the administered doses and the selected time points (FIG. 5).

No significant decrease of MMP for the 0.5× dose was observed in BJ cells. For cells treated with the phytocompound combination at the 1× and 2× bioavailable doses, a statistically significant decrease of MMP was observed, though it depended on the induction time. For the 1× dose a statistically significant decrease was observed after 24 h (when it reached its maximum) and after 72 h, when the observed MMP level was 40% of the control. For the 2× bioavailable dose, a decrease in MMP was observed after 24 h and 48 h, and then MMP increased to approximately 50% of the control level after 72 h. The results suggest that normal cells demonstrate a high potential to recover their full functionality.

A statistically significant increase of the cell membrane permeability in MOLT-4 neoplastic cells was observed for all doses of the phytocompound mix at all time points (FIG. 5). As for BJ cells, no statistically significant increase of the number of cells with increased cell membrane permeability was observed for doses 0.5× and 1× after 72 h, which indicates that these cells demonstrate lower sensitivity to the impact of the phytocompound mix at the specified doses and time points.

### Recapitulation

To recapitulate, a phytocompound mix (genistein, curcumin, quercetin, resveratrol) affects the functions of the mitochondria, observed as a decrease of the mitochondrial membrane potential. In MOLT-4 neoplastic cells the decrease of MMP was observed as early as after 24 h for all administered doses of the mix, where the degree of the decrease depended on the administered dose, irrespective of the induction time. For normal BJ cells, decreases in MMP were also observed (for doses 1× and 2×), though they reached their maximum levels only at the 24 h time point (dose 1×), and the 24 h and 48 h time points (2×). Less significant decreases of the MMP, combined with a relatively low number of cells demonstrating increased permeability of the cell membrane for normal cells at the 1× and 2× doses at the time point of 72 h, may signify a weaker effect of the analysed phytocompound mix on those cells.

In addition, the performed experiments enabled the development of an optimal composition of the mix of the phytocompounds characterised by anti-cancer properties, necessary to attain the effective anti-cancer effects.

What is claimed is:

1. A composition of the mix of the following phytocompounds: curcumin, genistein, resveratrol, quercetin applied to enhance cytotoxic activity in the treatment of acute lymphoblastic leukemias.

2. The composition according to claim 1, characterised in that the cytotoxic activity of a specific compound in blood is 2.25 μg/ml for curcumin, 3 μg/ml for genistein, 1.5 μg/ml for quercetin, and 0.5 μg/ml for resveratrol.

3. The composition according to claim 1, characterised in that it is intended for paediatric patients.

4. The composition according to claim 1, characterised in that it is administered orally.
